(19) United States
(12) Patent Application Publication
(10) Pub. No.: US 2012/0143083 A1
(43) Pub. Date: Jun. 7, 2012

(54) DEVICES AND METHODS FOR IMPROVING THE USABILITY OF STEREOTACTIC IMAGING FOR PERFORMING A BREAST BIOPSY

(76) Inventor: Andrew Kwai, New York City, NY (US)

(21) Appl. No.: 12/958,283

(22) Filed: Dec. 1, 2010

(51) Int. Cl. A61B 10/02

(52) U.S. Cl. 600/567

(57) ABSTRACT

A device to distance a breast of a patient between a breast support plate and a compression plate while undergoing a stereotactic biopsy comprises a platform distanced from the breast support plate by a distancing structure connected with the platform when the device is mounted on the breast support plate. The device can be bound to the breast support plate to resist movement of the platform relative to the breast support plate. The platform is formed of a material that permits x-rays to pass through the platform substantially unobstructed such that no artifacts are introduced by the platform during imaging.

### TECHNICAL FIELD

This invention relates generally to imaging and obtaining biopsy samples from a patient's breast.

### BACKGROUND

Many impalpable abnormalities have imaging features that cannot definitely be diagnosed benign based on a mammogram, but rather they require some form of tissue diagnosis. Several techniques for performing a breast biopsy now exist. The most appropriate technique for a patient depends upon a variety of factors, including the size, location, appearance and characteristics of the abnormality. Minimally invasive stereotactic biopsies have proven to be accurate, cheap and patient friendly procedures. However, the location of the abnormality (referred to hereinafter as a lesion) and/or the size and shape of the breast may reduce the accuracy or appropriateness of stereotactic techniques.

### BRIEF DESCRIPTION OF THE DRAWINGS

FIG. 1A-1D is a side view of a breast positioned for imaging the breast and performing an upright stereotactic biopsy in accordance with the prior art.

FIG. 2A is a top view of an embodiment of a device in accordance with the present invention for elevating a breast undergoing a stereotactic biopsy.

FIG. 2B is a cross-section of the device of FIG. 2A.

FIG. 2C is a top perspective view of the device of FIG. 2A.

FIG. 2D is a bottom perspective view of the device of FIG. 2A.

FIG. 3 is a side view of an embodiment of a system in accordance with the present invention for performing a stereotactic biopsy on a breast of a patient in a generally upright position using the device of FIG. 2A.

FIG. 4 is a side view of an alternative embodiment of a system and device in accordance with the present invention for performing a stereotactic biopsy on a breast of a patient in a generally upright position.

FIG. 5 is a side view of a further embodiment of a system in accordance with the present invention for performing a stereotactic biopsy on a breast of a patient in a generally upright position using the device of FIG. 2A.

FIG. 6 is a side view of an alternative embodiment of a system in accordance with the present invention for performing a stereotactic biopsy on a breast of a patient in a generally prone position using the device of FIG. 2A.

FIG. 7 is a flowchart of an embodiment of a method in accordance with the present invention for performing a stereotactic biopsy on a breast of a patient in a generally upright position.

### DETAILED DESCRIPTION OF THE DRAWINGS

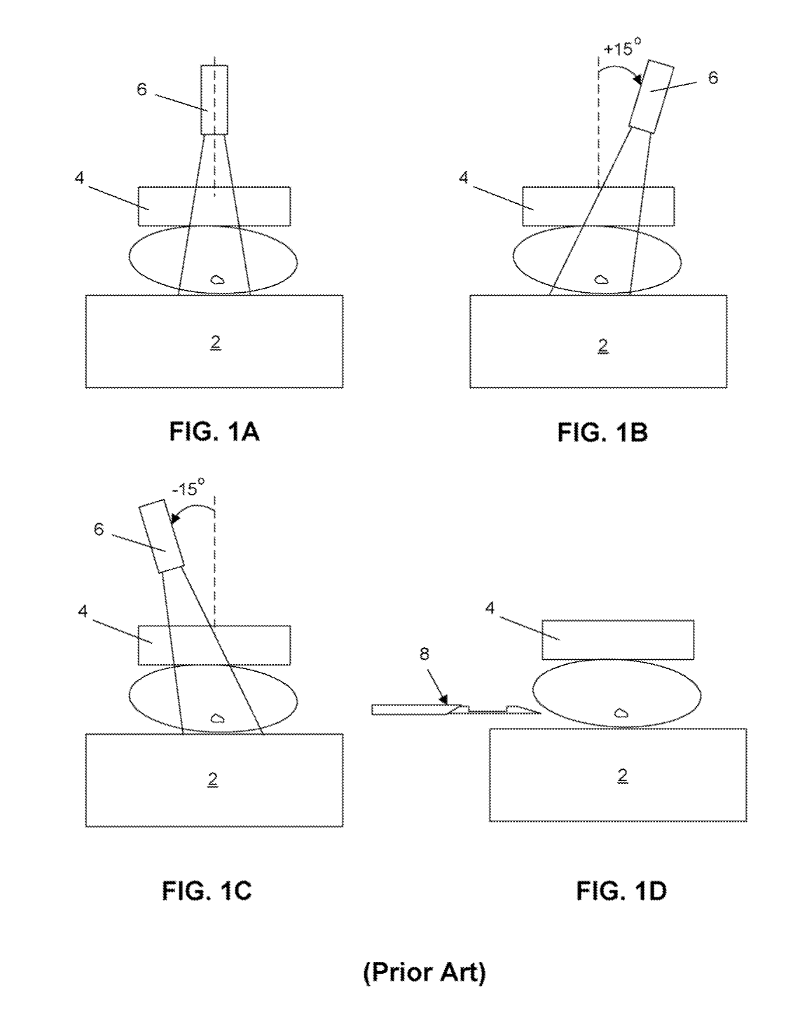

FIGS. 1A-1D are side views illustrating a technique in accordance with the prior art for imaging a breast to identify the location of a lesion and for performing a breast biopsy based on the identified location to obtain a sample of the lesion. The technique shown, known as an upright stereotactic biopsy, relies on stereotactic positioning in which a pair of stereo images is generated to calculate the location of the lesion in X-, Y-, and Z-coordinates based on the relative movement of the lesion in each image compared to a fixed reference point. The breast is prepared by supporting the breast on a breast support plate 2, which can be an imaging plate, of the stereotactic imaging machine. Examples of upright stereotactic imaging machines include the DELTA 32™ stereotactic unit manufactured by GENERAL ELECTRIC®, Co. and the STEREOLOC® II system manufactured by HOLOGIC®, Inc. The breast is then compressed against the breast support plate 2 using a compression plate 4 to provide better resolution. As shown in FIGS. 1B and 1C, the stereo images are generated at +15° and −15° from vertical using an x-ray device 6, although other angles can be chosen.

Once the coordinates are calculated, a biopsy gun having core needle 8 can be manually positioned and inserted into the breast or placed proximate to the breast by a clinician. The core needle 8 has a special cutting edge allowing removal of a relatively large sample of tissue through a small incision in the skin. Typically, the breast area is first locally anesthetized with a small amount of anesthetic fluid. Then, the core needle 8 is placed close to or into the breast. The core biopsy gun is then “fired” so that the cutting edge extends from a hollow needle to penetrate the breast to the lesion so that a portion of the lesion relapses or is otherwise held within a pocket distal of the cutting edge. The hollow needle then follows and sheaths the pocket to cut and capture the sample within the pocket. The core needle 8 is then removed from the breast so that the sample can be analyzed.

Problematically, the prior art technique can result in relative positioning of the lesion and the core needle such that the core needle cannot safely enter the lesion. For example, one such unsafe condition can result if the lesion is located in a more inferior aspect of the breast within a breast below a certain thickness. With many biopsy guns on the market, there is a minimum height that the lesion must be from the breast support plate for the core needle to have adequate clearance to enter the breast tissue. This minimum height may be biopsy gun specific, and may be caused by the size and shape of components other than the core needle, such as the biopsy gun handle. For example, clearance can be problematic with the HOLOGIC® Inc. manufactured SUROS® branded core needles commonly used in stereotactic biopsies, which can require, for example, 2.5 mm of clearance. Embodiments of devices in accordance with the present invention can be used to elevate the breast of a patient between the breast support plate and the compression plate in a way that does not interfere with positioning of a biopsy gun.

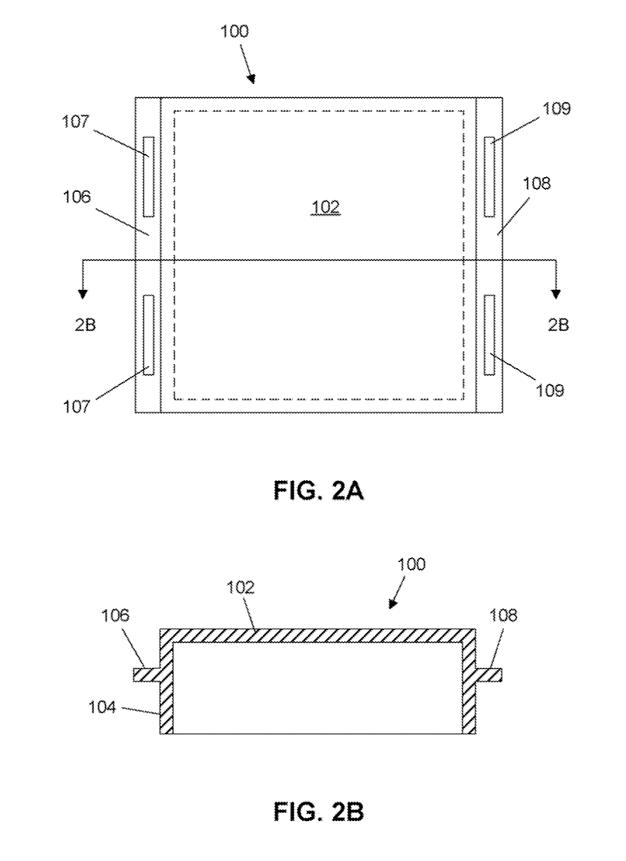

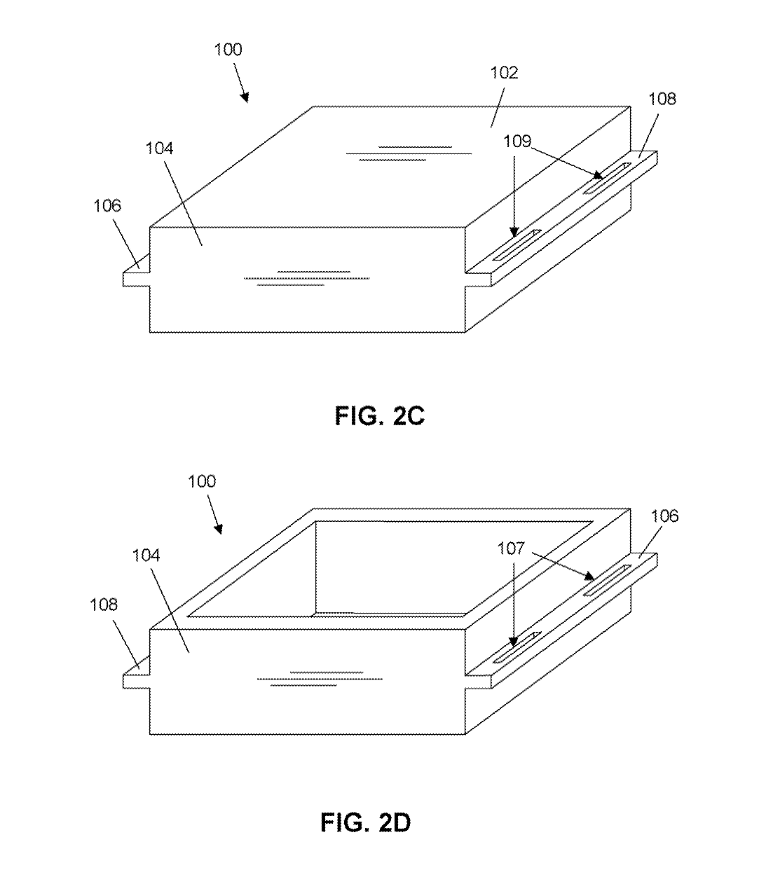

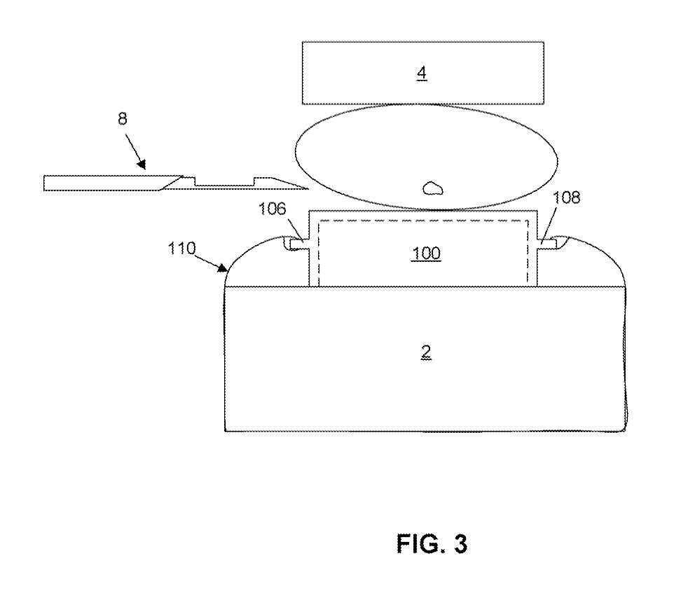

Referring to FIGS. 2A-3, one such embodiment is shown comprising a device 100 with a platform 102 distanced from the breast support plate 2 of a stereotactic imaging machine when the device 100 is positioned on the breast support plate 2 by a distancing structure 104 connected with the platform 102. As shown, the platform 102 is roughly square in footprint and the distancing structure 104 is formed by a contiguous wall that extends down from the sides of the platform 102. In other embodiments, the distancing structure need not be formed by a contiguous wall, but can be formed by some other arrangement of structures capable of stably supporting the platform, for example the distancing structure can include three or more legs. Further, the distancing structure need not be integrally formed with the platform or formed from the same material as the platform, but rather can be separately formed of the same or different material and attached to the platform. In still other embodiments, the platform can have a footprint having some other shape, such as rectangular or circular. Preferably the device 100 is sized and shaped so that the device 100 sits stably on the breast support plate and can satisfactorily support the patient's breast.

The platform 102 comprises a material that permits imaging without introducing potentially problematic artifacts. Where imaging is performed using x-rays, the platform 102 comprises a material that permits x-rays to pass through the platform 102 substantially unobstructed such that no artifacts are introduced by the platform 102, or such that or a negligible amount of artifacts are introduced by the platform 102. In one embodiment, the platform can be fabricated from the same material used to fabricate the compression plate. For example, in one embodiment the platform 102 is fabricated from clear acrylic polymer. However, in other embodiments the platform can be fabricated from some other material, the same or different from a material used to fabricate the compression plate, so long as the material is acceptably transparent to the imaging device. Further, the platform 102 and a distancing structure 104 can have a thickness that is sufficiently capable of supporting a breast and the weight of compression of the breast against the platform 102, while being thin relative to the platform height so as to further reduce the possibility of introducing artifacts into an image. Thus, the space between the top surface of the platform 102 and the top surface of the breast support plate is preferably as empty as is practicable. For example, in one embodiment, the device includes a square platform that is 12 cm in width, 12 cm in length, and 0.5 cm in thickness, and that is distanced to a height of 2.5 cm above the breast support plate by a wall that is 0.5 cm thick. However, one of ordinary skill in the art, upon reflecting on the teachings provided herein, will appreciate the myriad different shapes, sizes, and dimensions with which embodiments of devices in accordance with the present invention can be formed.

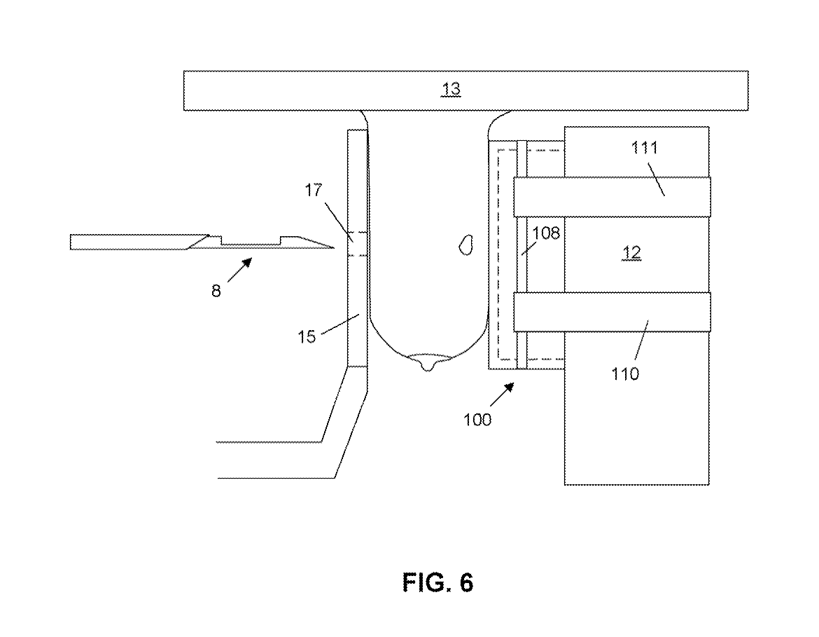

As shown in the cross-section of FIG. 2B and the perspective views of FIGS. 2C and 2D, the device 100 further includes ledges 106, 108 arranged on opposite sides of the platform 102 integrally formed or fixedly attached with a distancing structure 104. Each ledge 106, 108 includes at least one slit or anchor point for attaching a binder (110 in FIGS. 3, and 110, 111 in FIG. 6). In the embodiment shown, each ledge 106, 108 includes two slits 107, 109 through which the binder 110 is inserted and fixed in place, for example by looping the binder through the slit and secured to itself on the other side of the binder 110. In the exemplary embodiment described above, the ledges can be 0.5 cm in height, 1 cm in width, and include slits that are 3 cm in length. However, in other embodiments any number of slits having the same or different lengths can be formed in the ledge, or alternatively an anchor structure other than a ledge can be used as an anchor point, such as one or more eyelet bolts attached to the distancing structure. The anchor structure, whether a ledge with slots, or some other some other structure need only allow for the attachment of a binder without introducing artifacts into the stereo images used for calculating lesion position.

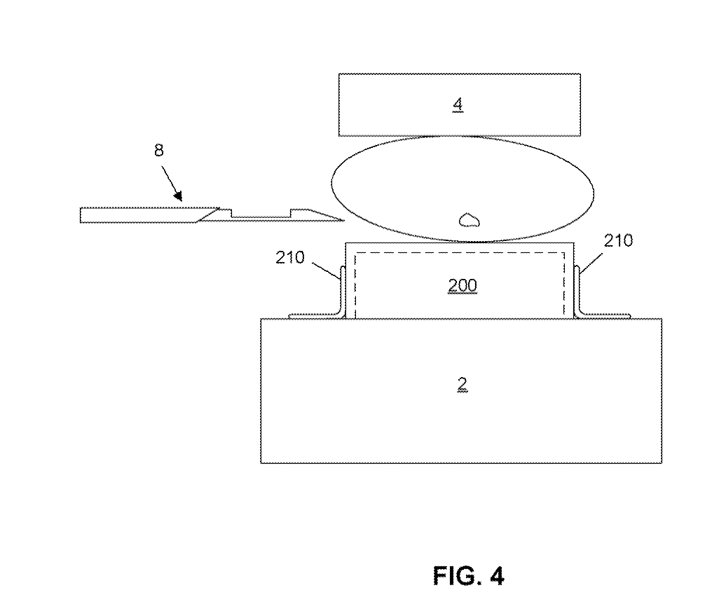

In still further embodiments, the distancing structures themselves can function as anchor points. For example, referring to FIG. 4, an alternative embodiment of a device 200 in accordance with the present invention can be secured to the breast support plate 2 by adhesive tape 210 adhered to the wall 204 of the device 200 and adhered to a portion of the breast support plate 2 extending beyond the footprint of the device 200. In still other embodiments, the device can be adhered to the surface of the breast support plate, for example by a temporary adhesive, such as dissolvable glue.

Referring to FIG. 4, an embodiment of a system in accordance with the present invention for performing a stereotactic biopsy on a breast of a patient in a generally upright position comprises the device 100 and a binder 110 connected to opposite sides of the device 100 by way of ledges 106, 108. The binder 110 wraps around the breast support plate 2 and resists movement of the platform 102 relative to the breast support plate 2. The binder 110 is preferably a flexible strap, allowing the binder 110 to accommodate different sized and shaped breast support plates. In some embodiments, the binder 110 can be elastic so that the binder 110 can urge the device 100 against the breast support plate 2. Additionally, or alternatively, the length of the binder 110 can be adjustable so that the binder 110 applies a desired force to urge the device 100 against the breast support plate 2. In the exemplary embodiment described above, the binder can include two pairs of VELCRO® straps, each strap having a length, for example, of 84 cm. However, in other embodiments, the binder can be any device (or devices) attachable to an anchor point of the device and associable with the breast support plate of the stereotactic machine so as to resist relative movement of the device and the breast support plate.

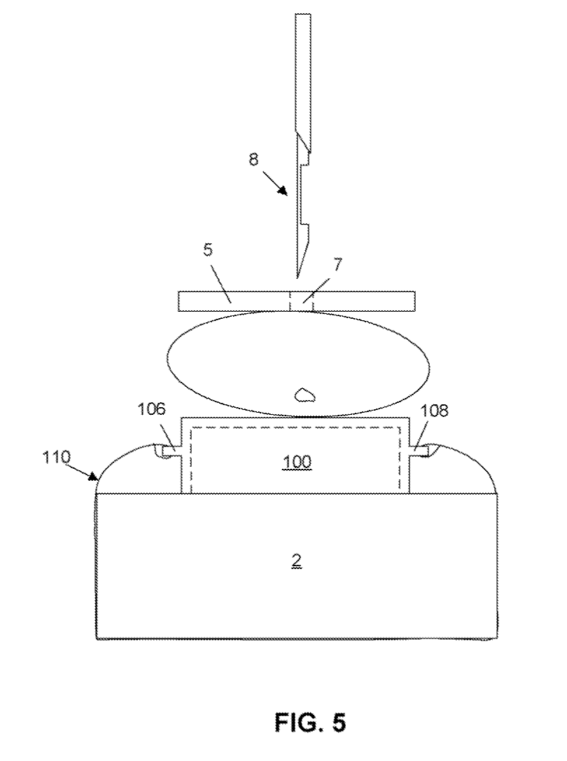

The technique for positioning and firing a biopsy gun described above includes a lateral approach to the breast with the core needle. Referring to FIG. 5, in other embodiments, a biopsy gun can be positioned for a vertical approach to the breast. When used for a vertical approach, the imaging machine may prevent a biopsy from being performed due to safety limits inherent to the system. Embodiments of devices and systems in accordance with the present invention can be applied to elevate the breast of the patient between a breast support plate 2 and a compression plate 5 while undergoing a stereotactic biopsy in a generally upright position, so that the elevation artificially increases the thickness of the breast as perceived by the stereotactic imaging machine, thereby misleading the stereotactic imaging machine to permit new coordinates to be generated for performing the biopsy and overcoming minimum distances required to overcome safety settings. The compression plate 5 in such an embodiment can have one or more perforations 7 through which the core needle 8 can access the breast.

Devices in accordance with the present invention can also be used with systems in which the patient is positioned face down (i.e., prone) with the breast extending generally downward through an opening in a specially designed table. An example of a prone stereotactic imaging system can include a STEREOLOC® II imaging unit combined with a MULTICARE™ prone breast biopsy table, both manufactured by HOLOGIC®, Inc. Referring to FIG. 6, embodiments of devices and systems in accordance with the present invention are shown for performing a stereotactic biopsy on a breast of a patient in a generally face down position. As can be seen, the device 100 is secured to a vertically arranged breast support plate 12 to expand a distance between a vertically arranged compression plate 15 and the breast support plate 12. The patient's breast is positioned through an opening in the table 13 and is compressed between the compression plate 15 and the breast support plate 12 for imaging and biopsy. As above, the compression plate 15 in such an embodiment can have one or more perforations 17 through which the core needle 8 can access the breast. Such an arrangement can provide a similar benefit that described above, in that the distance between the plates artificially increases the thickness of the breast as perceived by the stereotactic imaging machine, thereby misleading the stereotactic imaging machine to permit new coordinates to be generated for performing the biopsy. As shown, the device 100 is prevented from shifting in place or detaching from the vertical breast support plate 12 by binders 110, 111, although as described above, in other embodiments some other anchoring technique can be used. However, devices 100 used with embodiments of systems shown in FIG. 6 may benefit from use of binders 110, 111 that are less elastic than those used in the embodiments shown in FIGS. 3-5, and/or capable of apply sufficient compressive force to the device 110 to urge the device 110 against the vertical breast support plate 12 and resist relative movement.

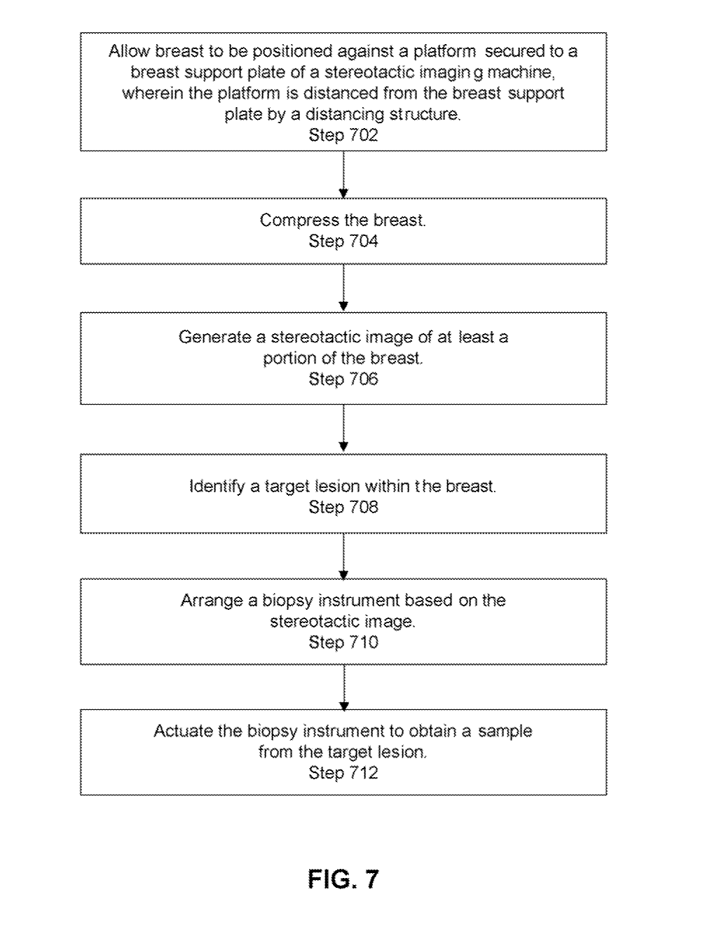

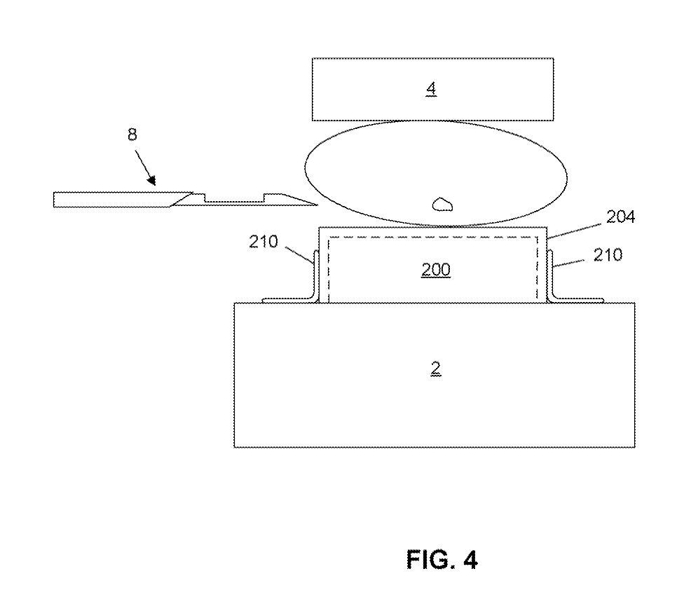

FIG. 7 is a flowchart illustrating a method of obtaining a sample from a lesion in a breast of a patient sitting in a generally upright position. The method comprises allowing the breast of the patient to be positioned on a platform of a device secured to a breast support plate of a stereotactic imaging device (Step 702). Alternatively, where the method is applied to a biopsy performed on a prone patient, the breast of the patient is inserted through an opening in a biopsy table and abutted against the platform of the device secured to the breast support plate. The platform is distanced from the breast support plate by a distancing structure. The breast is compressed (Step 704), for example by a compression plate. At least a pair of stereotactic images of at least a portion of the breast is then generated (Step 706). A target lesion within the breast is identified from the stereotactic images (Step 708). A biopsy instrument is arranged based on the stereotactic image (Step 710) and the biopsy instrument is actuated to obtain a sample from the target lesion (Step 712). The method can further comprise positioning the device on or against the breast support plate and securing the platform to the breast support plate using a binder. Where the biopsy instrument is a biopsy gun with a core needle, the needle can be manually positioned within a small incision of the breast, or alternatively proximate to the breast. The biopsy gun is actuated by discharging the biopsy gun so that a cutting edge of the core needle penetrates the target lesion and a hollow needle then advances to cut and capture a sample of the target lesion. Once the sample is captured, the core needle can be withdrawn from the breast, and the sample can be retrieved and inspected.

Devices, systems and methods for obtaining samples from a target lesion have been described above as for use with, using, or including core needles for obtaining the samples. However, embodiments of devices, systems and methods in accordance with the present invention are not intended to be restricted to core needles. For example, in other embodiments, devices, systems and methods can obtain samples using a vacuum assisted biopsy technique. Still further, devices, systems and methods have been described above as for use with, using or including stereotactic imaging using x-rays. However, embodiments of devices, systems and methods in accordance with the present invention are not intended to be restricted to imaging using x-rays. For example, in other embodiments, devices, systems and methods can be used with, or use images or location information obtained by ultrasonography, or magnetic resonance imaging (MRI).

The foregoing description of the present invention has been presented for purposes of illustration and description. It is not intended to be exhaustive or to limit the invention to the precise forms disclosed. Many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

1. A device to distance a breast of a patient between a breast support plate and a compression plate while undergoing a stereotactic biopsy, comprising:
a platform distanced from the breast support plate by a distancing structure connected with the platform when the device is mounted on the breast support plate;
wherein the platform is formed of a material that permits x-rays to pass through the platform substantially unobstructed such that no artifacts are introduced by the platform during imaging.

2. The device of claim 1, further comprising:
a binder to secure the platform so as to resist movement of the platform relative to the breast support plate;
wherein the distancing structure include a first anchor point for attaching the binder at a first end and a second anchor point for attaching the binder at a second end;

3. The device of claim 2, wherein:
the platform has an approximately rectangular footprint; and
the distancing structure include a wall extending from each of four sides of the platform;
the wall includes a first ledge extending from a first side of the wall and a second ledge extending from a second side of the wall opposite the first side; and
the first anchor point is one or more slits formed in the first ledge and the second anchor point is one or more slits formed in a second ledge.

4. The device of claim 3, wherein the binder includes one or more flexible or semi-flexible straps inserted through the one or more slits of the first ledge and the second ledge.

5. The device of claim 1, wherein the platform and the walls are integrally formed from clear acrylic polymer.

6. A system for performing a stereotactic biopsy on a breast of a patient, comprising:
a stereotactic imaging machine including a breast support plate;
a platform distanced from the breast support plate by a distancing structure connected with the platform when the device is mounted on the breast support plate;
a compression plate to compress the breast against the platform;
wherein the platform is formed of a material that permits x-rays to pass through the platform substantially unobstructed such that no artifacts are introduced by the platform during imaging; and
a biopsy instrument positionable in response to a stereotactic image to obtain a sample from a target lesion.

7. The system of claim 6, wherein the breast support plate is an imaging plate.

8. The device of claim 6, further comprising:
a binder to resist movement of the platform relative to the breast support plate;
wherein the distancing structure include a first anchor point for attaching the binder at a first end and a second anchor point for attaching the binder at a second end;

9. The system of claim 8, wherein:
the platform has an approximately rectangular footprint; and
the distancing structure include a wall extending from each of four sides of the platform;
the wall includes a first ledge extending from a first side of the wall and a second ledge extending from a second side of the wall opposite the first side; and
the first anchor point is one or more slits formed in the first ledge and the second anchor point is one or more slits formed in a second ledge.

10. The device of claim 9, wherein the binder includes one or more flexible or semi-flexible straps inserted through the one or more slits of the first ledge and the second ledge.

11. The system of claim 6, wherein the platform and the walls are integrally formed from clear acrylic polymer.

12. The system of claim 6, wherein the stereotactic imaging machine is one of a DELTA 32™ stereotactic unit manufactured by GENERAL ELECTRIC® and a STEREOLOC® II system manufactured by HOLOGIC®.

13. The system of claim 6, wherein the biopsy instrument is a core needle.

14. A method of obtaining a sample from a lesion in a breast of a patient sitting in a generally upright position, comprising:
allowing the breast to be positioned against a platform secured to a breast support plate of a stereotactic imaging machine, wherein the platform is distanced from the breast support plate by a distancing structure;
compressing the breast;
generating at least a pair of stereotactic images of at least a portion of the breast;
identifying a target lesion within the breast based on the stereotactic images;
arranging a biopsy instrument based on the stereotactic images; and
actuating the biopsy instrument to obtain a sample from the target lesion.

15. The method of claim 14, further comprising:
securing the platform to the breast support plate using a binder.

16. The method of claim 14, wherein the generated stereotactic image is substantially free of artifacts related to the platform.

17. The method of claim 14, further comprising:
positioning a needle of the biopsy instrument near the target lesion; and
wherein actuating the biopsy instrument includes discharging the biopsy instrument so that a cutting edge penetrates the target lesion before an outer needle advances to cut and capture the sample.

18. The method of claim 14, further comprising:
inspecting the sample.

19. The method of claim 14, wherein:
generating the stereotactic images are obtained at different angles; and
calculating the location of the target lesion in x-, y-, and z-coordinates based on the stereotactic image.

20. A device to distance a breast of a patient between a breast support plate and a compression plate while undergoing a stereotactic biopsy in a generally upright position, comprising:
a platform having an approximately rectangular footprint, the platform being distanced from the breast support plate by a wall extending from the platform when the device is mounted on the breast support plate;
wherein the platform and the wall are integrally formed from a clear acrylic polymer;
a flexible binder to resist movement of the platform relative to the breast support plate;
a first anchor point at a first side of the wall for attaching the binder at a first end and a second anchor point at a second side of the wall opposite the first side for attaching the binder at a second end;
wherein the platform permits x-rays to pass through the platform substantially unobstructed such that no artifacts are introduced by the platform during imaging.
